(19) United States
(12) Patent Application Publication
(10) Pub. No.: US 2022/0112350 A1
(43) Pub. Date: Apr. 14, 2022

(54) FOAM CASTING PROCESS

(71) Applicant: Endoluminal Sciences Pty Ltd, Eveleigh (AU)

(72) Inventor: Ashish MITRA, Bondi (AU)

(21) Appl. No.: 17/559,956

(22) Filed: Dec. 22, 2021

Related U.S. Application Data
(63) Continuation of application No. PCT/IB2020/000540, filed on Jun. 26, 2020.
(60) Provisional application No. 62/867,159, filed on Jun. 26, 2019.

(51) Int. Cl. C08J 9/228; B29C 44/04; B29C 44/34; C08J 9/00

(52) U.S. Cl. CPC C08J 9/228; B29C 44/0484; B29C 44/355; C08J 9/0066; C08J 9/0023; C08J 2205/052; C08J 2375/04; C08J 2207/10; C08J 2205/044; C08J 2205/05; C08J 2201/022

(57) ABSTRACT

Disclosed herein are manufacturing/casting processes for the preparation of a foam.

### CROSS-REFERENCE

This application is a continuation of Application No. PCT/IB2020/000540, filed Jun. 26, 2020, which is incorporated herein by reference in its entirety and to which application we claim priority under 35 USC § 120; and this application claims the benefit of U.S. Provisional Application No. 62/867,159, filed Jun. 26, 2019, which application is incorporated herein by reference.

### BACKGROUND OF THE INVENTION

The present invention relates to a casting process of a foam that allows fabrication/manufacturing of foams with customized and potentially highly complex/intricate shapes. The foams created by this process may be used for permanent implantation in the human body while in contact with blood, and therefore may be made from highly biocompatible and biostable materials. The process allows highly expandable and/or compressible foams to be produced, which can be delivered in the human body by compressing in a catheter and expanded to a predetermined shape once implanted. The process further allows for integration of additional materials/components such as a mesh or a film with the foam.

### SUMMARY OF THE INVENTION

Described herein are manufacturing/casting processes for the preparation of a foam. Advantageously, the foam manufacturing/casting processes described herein use a combination of a salt-leaching and solvent casting/washing/evaporation technique. Another advantage of the foam manufacturing/casting processes described herein includes the synthesis of foams using pre-polymerized polymer pellets in the absence of an isocyanate-polyol reaction. In some embodiments of the foam manufacturing/casting processes described herein foams are synthesized using polyurethane in the polymer state. In some embodiments, the processes described herein allow for the manufacture of foams using a co-solvent. In some embodiments, the foam manufacturing/casting processes described herein allow for the manufacture of foams using a co-solvent that uses a combination of solvent removal techniques. An additional advantage of the foam manufacturing/casting processes described herein includes the ability to cast foams into a predetermined shape at a millimeter scale. A further advantage of the foam manufacturing/casting processes described herein includes the ability to manufacture foams with materials other than polyurethane. For example, materials which are not water soluble, but soluble in particular organic solvents. A further advantage of the foam manufacturing/casting processes described herein includes the ability to manufacture foams with a controlled porosity, wherein the porosity is controlled at least by the amount of solid particulate added to the polymer solution in the mold, the size of the solid particulate, and/or the concentration of the polymer solution in the mold. In some embodiments, the foam is manufactured with a controlled porosity which is variable throughout the structure of the foam. In some embodiments, the foam is manufactured with a gradient of porosity throughout the structure of the foam. A further advantage of the foam manufacturing/casting processes described herein includes the ability to attach a mesh or membrane to the foam as part of the casting process. In some embodiments, a polyethylene terephthalate (PET) mesh is attached to the foam as part of the casting process.

In some embodiments, the foams described herein are used foam-based expandable sealing skirts for use with an endovascular prosthesis in a human patient. In some embodiments, the foams described herein are used foam-based expandable sealing skirts for use with an endovascular prosthesis in a human patient, wherein the endovascular prosthesis is a transcatheter valve implantation device. In some embodiments, the foams described herein are used foam-based expandable sealing skirts for use with an endovascular prosthesis in a human patient, wherein the endovascular prosthesis is a transcatheter aortic valve implantation device. In some embodiments, the foams described herein are used foam-based expandable sealing skirts for use with an endovascular prosthesis in a human patient, wherein the endovascular prosthesis is an endovascular stent graft.

In one aspect, disclosed herein is a process for the preparation of a foam, the process comprising a) dissolving a polymer material in a solvent to form a polymer solution; b) adding the polymer solution to a mold, and optionally degassing the polymer solution in the mold; c) adding a solid particulate to the top of the polymer solution in the mold; and d) washing the resulting polymer mixture in the mold with a washing liquid; wherein the solid particulate is insoluble in the polymer solution and soluble in the washing liquid, and wherein after washing the polymer mixture with the washing liquid, the remaining polymer material in the mold forms the foam and the shape of the foam is defined by the shape of the mold. In some embodiments, the process further comprises forming a thin layer of polymer material on the mold before adding the polymer solution to the mold. In some embodiments, the concentration of the polymer solution is from 0.5%-25.0% (w/v). In some embodiments, the process further comprises placing a preheated PET mesh on top of the mold and heating the mold at 30-90° C. for up to 1 hour, after adding the solid particulate to the top of the polymer solution in the mold and before washing the resulting polymer mixture in the mold with the washing liquid. In some embodiments, the mold is preheated to a temperature from about 40-100° C. prior to the addition of the polymer solution to the mold. In some embodiments, the mold is preheated to a temperature from about 40-100° C. for at least 20 minutes. In some embodiments, the polymer solution is at room temperature, or preheated to a temperature from about 30-70° C., upon adding the polymer solution to the mold. In some embodiments, the polymer solution is added to the mold until it overflows. In some embodiments, the polymer solution in the mold is degassed. In some embodiments, the polymer solution in the mold is degassed in a vacuum oven. In some embodiments, the polymer solution in the mold is degassed in a vacuum oven at about 100 mbar-500 mbar. In some embodiments, the polymer solution in the mold is degassed in a vacuum oven at about 30-90° C. In some embodiments, the polymer solution in the mold is degassed in a vacuum oven at about 30-90° C. for up to 1 hour. In some embodiments, the polymer solution in the mold is not degassed. In some embodiments, the solid particulate is added to the top of the polymer solution in the mold in a ratio of 20-200 w/v. In some embodiments, the solid particulate particle size is between about 5-500 μm. In some embodiments, following the addition of the solid particulate to the top of the polymer solution in the mold and before washing the resulting polymer mixture in the mold with the washing liquid, the solvent is removed under vacuum. In some embodiments, following the addition of the solid particulate to the top of the polymer solution in the mold and before washing the resulting polymer mixture in the mold with the washing liquid, the solvent is removed in a vacuum oven at about 30-90° C. In some embodiments, following the addition of the solid particulate to the top of the polymer solution in the mold and before washing the resulting polymer mixture in the mold with the washing liquid, the solvent is removed in a vacuum oven at about 30-90° C. for at least 12 hours. In some embodiments, washing the resulting polymer mixture in the mold with the washing liquid comprises immersing the mold in the washing liquid. In some embodiments, washing resulting polymer mixture in the mold with the washing liquid comprises immersing the mold in the washing liquid, wherein the washing liquid temperature is from about 30-60° C. In some embodiments, the washing liquid is water. In some embodiments, the solvent is selected from dimethyl sulfoxide (DMSO), tetrahydrofuran (THF), dimethylformamide (DMF), dimethylacetamide (DMAc), and a combination thereof. In some embodiments, the solvent is DMSO. In some embodiments, the solvent is THF. In some embodiments, the solvent is DMF. In some embodiments, DMAc. In some embodiments, the solvent is a combination of DMSO and THF. In some embodiments, the polymer material is pre-polymerized polymer pellets. In some embodiments, the polymer material is polyurethane pellets. In some embodiments, the solid particulate is sodium chloride. In some embodiments, the solid particulate is sugar. In some embodiments, the foam has a controlled porosity. In some embodiments, the foam has a uniform porosity. In some embodiments, the foam has a variable porosity. In some embodiments, the foam has an open pore structure, a closed pore structure, or a combination. In some embodiments, the foam has a porosity of about 5-500 μm. In some embodiments, the foam is cast into a predetermined shape at a millimeter scale based on the shape of the mold. In some embodiments, the shape of the mold is selected from a triangular pyramid, square pyramid, oval, partial oval, spherical, partially spherical, rhomboid, diamond, partial diamond, and a customized 3-dimensional shape; or a combination thereof. In some embodiments, the mold is a silicon mold, a Teflon mold, or an aluminum mold. In some embodiments, the foam is used as a foam-based expandable sealing skirt for use with an endovascular prosthesis in a human patient. In some embodiments, the endovascular prosthesis is a transcatheter valve implantation device. In some embodiments, the endovascular prosthesis is a transcatheter aortic valve implantation device. In some embodiments, the endovascular prosthesis is an endovascular stent graft.

### BRIEF DESCRIPTION OF THE DRAWINGS

A better understanding of the features and advantages of the present subject matter will be obtained by reference to the following detailed description that sets forth illustrative embodiments and the accompanying drawings.

FIG. 1 depicts the preparation of a polymer solution of polyurethane pellets in a solvent by placing the pellets and solvent in a pyrex bottle and mixing with a magnetic stirrer.

FIG. 2 depicts the casting of a silicon mold.

FIG. 3 depicts the addition of polymer solution to the mold via a pipette.

FIG. 4 depicts the addition of salt on top of the polymer solution in the mold.

FIG. 5 depicts the immersion of the mold in a water bath.

FIG. 6 depicts the addition of a PET mesh on top of the mold.

### DETAILED DESCRIPTION

Disclosed herein are manufacturing/casting processes for the preparation of a foam. In some embodiments, the manufacturing/casting process for the preparation of a foam comprises a) dissolving a polymer material in a solvent to form a polymer solution; b) adding the polymer solution to a mold; c) adding a solid particulate to the top of the polymer solution in the mold; and d) washing the polymer mixture in the mold with a washing liquid. In some embodiments, the manufacturing/casting process for the preparation of a foam comprises a) dissolving a polymer material in a solvent to form a polymer solution; b) adding the polymer solution to a mold; c) degassing the polymer solution in the mold; d) adding a solid particulate to the top of the polymer solution in the mold; and e) washing the polymer mixture in the mold with a washing liquid. In embodiments of the processes described herein, the solid particulate is insoluble in the polymer solution and soluble in the washing liquid, and after washing the polymer mixture with the washing liquid, the remaining polymer material in the mold forms the foam and the shape of the foam is defined by the shape of the mold.

### Preparation of Polymer Solutions

In embodiments of the processes described herein, a step in the process is the preparation of polymer solutions. In embodiments, pre-polymerized polymer pellets are dissolved in a solvent. In embodiments, pre-polymerized polymer pellets are dissolved in an organic solvent. In embodiments, pre-polymerized polymer pellets are dissolved in an organic solvent selected from dimethyl sulfoxide (DMSO), tetrahydrofuran (THF), dimethylformamide (DMF), dimethylacetamide (DMAc), dichloromethane, chloroform, and hexafluoroisopropanol (HFIP) or a combination thereof. In embodiments, pre-polymerized polymer pellets are dissolved in an organic solvent selected from dimethyl sulfoxide (DMSO), tetrahydrofuran (THF), dimethylformamide (DMF), and dimethylacetamide (DMAc), or a combination thereof. In some embodiments, pre-polymerized polymer pellets are dissolved in DMSO. In some embodiments, pre-polymerized polymer pellets are dissolved in THF. In some embodiments, pre-polymerized polymer pellets are dissolved in DMF. In some embodiments, pre-polymerized polymer pellets are dissolved in DMAc. In some embodiments, pre-polymerized polymer pellets are dissolved in dichloromethane. In some embodiments, pre-polymerized polymer pellets are dissolved in chloroform. In some embodiments, pre-polymerized polymer pellets are dissolved in HFIP. In some embodiments, pre-polymerized polymer pellets are dissolved in a combination of at least two organic solvents. In some embodiments, pre-polymerized polymer pellets are dissolved in a combination of two organic solvents. In some embodiments, pre-polymerized polymer pellets are dissolved in a combination of two organic solvents selected from DMSO, THF, DMF, DMAc, dichloromethane, chloroform, and HFIP. In some embodiments, pre-polymerized polymer pellets are dissolved in a combination of two organic solvents selected from DMSO, THF, DMF, and DMAc. In some embodiments, pre-polymerized polymer pellets are dissolved in a combination of DMSO and THF.

In some embodiments, pre-polymerized polymer pellets are dissolved in a solvent wherein the pre-polymerized polymer pellets are polyurethane pellets. In some embodiments, polyurethane pellets are dissolved in an organic solvent. In embodiments, polyurethane pellets are dissolved in an organic solvent selected from dimethyl sulfoxide (DMSO), tetrahydrofuran (THF), dimethylformamide (DMF), dimethylacetamide (DMAc), dichloromethane, chloroform, and hexafluoroisopropanol (HFIP) or a combination thereof. In embodiments, polyurethane pellets are dissolved in an organic solvent selected from dimethyl sulfoxide (DMSO), tetrahydrofuran (THF), dimethylformamide (DMF), and dimethylacetamide (DMAc), or a combination thereof. In some embodiments, polyurethane pellets are dissolved in DMSO. In some embodiments, polyurethane pellets are dissolved in THF. In some embodiments, polyurethane pellets are dissolved in DMF. In some embodiments, polyurethane pellets are dissolved in DMAc. In some embodiments, polyurethane pellets are dissolved in dichloromethane. In some embodiments, polyurethane pellets are dissolved in chloroform. In some embodiments, polyurethane pellets are dissolved in HFIP. In some embodiments, polyurethane pellets are dissolved in a combination of at least two organic solvents. In some embodiments, polyurethane pellets are dissolved in a combination of two organic solvents. In some embodiments, polyurethane pellets are dissolved in a combination of two organic solvents selected from DMSO, THF, DMF, and DMAc. In some embodiments, polyurethane pellets are dissolved in a combination of DMSO and THF.

In some embodiments, pre-polymerized polymer pellets are dissolved in a solvent wherein the pre-polymerized polymer pellets are polyethylene terephthalate (PET) pre-polymerized pellets. In some embodiments, PET pre-polymerized pellets are dissolved in an organic solvent. In embodiments, PET pre-polymerized pellets are dissolved in an organic solvent selected from trifluoroacetic acid or trichloroacetic acid. In some embodiments, PET pre-polymerized pellets are dissolved in trifluoroacetic acid. In some embodiments, PET pre-polymerized pellets are dissolved in trichloroacetic acid.

In some embodiments, the concentration of the polymer solution is from 0.5%-25.0% (w/v). In some embodiments, the concentration of the polymer solution is from 1.0%-25.0% (w/v). In some embodiments, the concentration of the polymer solution is from 2.0%-25.0% (w/v). In some embodiments, the concentration of the polymer solution is from 3.0%-25.0% (w/v). In some embodiments, the concentration of the polymer solution is from 4.0%-25.0% (w/v). In some embodiments, the concentration of the polymer solution is from 5.0%-25.0% (w/v). In some embodiments, the concentration of the polymer solution is from 5.0%-20.0% (w/v). In some embodiments, the concentration of the polymer solution is from 5.0%-15.0% (w/v). In some embodiments, the concentration of the polymer solution is from 0.5%-20.0% (w/v). In some embodiments, the concentration of the polymer solution is from 0.5%-15.0% (w/v). In some embodiments, the concentration of the polymer solution is from 0.5%-10.0% (w/v).

In some embodiments wherein the pre-polymerized polymer pellets are dissolved in a combination of two solvents, the ratio of the two solvents is from 1% to 99%. In some embodiments wherein the pre-polymerized polymer pellets are dissolved in a combination of two solvents, the ratio of the two solvents is from 5% to 95%. In some embodiments wherein the pre-polymerized polymer pellets are dissolved in a combination of two solvents, the ratio of the two solvents is from 10% to 95%. In some embodiments wherein the pre-polymerized polymer pellets are dissolved in a combination of two solvents, the ratio of the two solvents is from 15% to 95%. In some embodiments wherein the pre-polymerized polymer pellets are dissolved in a combination of two solvents, the ratio of the two solvents is from 20% to 95%. In some embodiments wherein the pre-polymerized polymer pellets are dissolved in a combination of two solvents, the ratio of the two solvents is from 25% to 95%. In some embodiments wherein the pre-polymerized polymer pellets are dissolved in a combination of two solvents, the ratio of the two solvents is from 10% to 90%. In some embodiments wherein the pre-polymerized polymer pellets are dissolved in a combination of two solvents, the ratio of the two solvents is from 10% to 80%. In some embodiments wherein the pre-polymerized polymer pellets are dissolved in a combination of two solvents, the ratio of the two solvents is from 15% to 75%. In some embodiments wherein the pre-polymerized polymer pellets are dissolved in a combination of two solvents, the ratio of the two solvents is from 20% to 75%. In some embodiments wherein the pre-polymerized polymer pellets are dissolved in a combination of two solvents, the ratio of the two solvents is from 25% to 75%.

### Addition of Polymer Solution to the Mold

In embodiments of the processes described herein, a step in the process is the addition of a polymer solution to the mold. In some embodiments, the mold is preheated prior to the addition of the polymer solution to the mold. In some embodiments, the mold is preheated to a temperature from about 40-100° C. prior to the addition of the polymer solution to the mold. In some embodiments, the mold is preheated to a temperature from about 40-100° C. for at least 10 minutes prior to the addition of the polymer solution to the mold. In some embodiments, the mold is preheated to a temperature from about 40-100° C. for at least 20 minutes prior to the addition of the polymer solution to the mold. In some embodiments, the mold is preheated to a temperature from about 40-100° C. for at least 30 minutes prior to the addition of the polymer solution to the mold. In some embodiments, the mold is preheated to a temperature from about 40-100° C. for at least 45 minutes prior to the addition of the polymer solution to the mold. In some embodiments, the mold is preheated to a temperature from about 40-100° C. for at least 1 hour prior to the addition of the polymer solution to the mold.

In some embodiments, a thin layer of polymer is formed on the surface of the mold prior to the addition of the polymer solution to the mold. In some embodiments, a thin layer of polymer is formed on the surface of the mold prior to the addition of the polymer solution to the mold, wherein the thin polymer layer is formed by adding a polymer solution to the mold and removing the solvent under reduced pressure, heat, or a combination thereof.

In some embodiments, the polymer solution is at room temperature, or preheated to a temperature from about 30-70° C., upon adding the polymer solution to the mold. In some embodiments, the polymer solution is at room temperature upon adding the polymer solution to the mold. In some embodiments, the polymer solution is preheated to a temperature from about 30-70° C. upon adding the polymer solution to the mold. In some embodiments, the polymer solution is preheated to a temperature from about 40-70° C. upon adding the polymer solution to the mold. In some embodiments, the polymer solution is preheated to a temperature from about 50-70° C. upon adding the polymer solution to the mold.

In some embodiments, the polymer solution is added to completely fill the mold. In some embodiments, the polymer solution is added to the mold until it overflows.

### Vacuum Degassing

In embodiments of the processes described herein, an optional step in the process is the degassing of the polymer solution in the mold. In some embodiments of the processes described herein, the polymer solution in the mold is degassed. In some embodiments, the polymer solution in the mold is degassed in a vacuum oven. In some embodiments, the polymer solution in the mold is degassed in a vacuum oven at about 100 mbar-500 mbar. In some embodiments, the polymer solution in the mold is degassed in a vacuum oven at about 100 mbar-400 mbar. In some embodiments, the polymer solution in the mold is degassed in a vacuum oven at about 100 mbar-300 mbar. In some embodiments, the polymer solution in the mold is degassed in a vacuum oven up to about 300 mbar. In some embodiments, the polymer solution in the mold is degassed in a vacuum oven at about 30-90° C. In some embodiments, the polymer solution in the mold is degassed in a vacuum oven at about 30-90° C. for up to 2 hours. In some embodiments, the polymer solution in the mold is degassed in a vacuum oven at about 30-90° C. for up to 1.5 hours. In some embodiments, the polymer solution in the mold is degassed in a vacuum oven at about 30-90° C. for up to 1 hour. In some embodiments, the polymer solution in the mold is degassed in a vacuum oven at about 30-90° C. for up to 30 minutes. In some embodiments, the polymer solution in the mold is degassed in a vacuum oven at about 100 mbar-500 mbar and at about 30-90° C. In some embodiments, the polymer solution in the mold is degassed in a vacuum oven at about 100 mbar-500 mbar and at about 30-90° C. for up to 2 hours. In some embodiments, the polymer solution in the mold is degassed in a vacuum oven at about 100 mbar-500 mbar and at about 30-90° C. for up to 1 hour. In some embodiments, the polymer solution in the mold is degassed in a vacuum oven up to about 300 mbar and at about 30-90° C. for up to 1 hour.

In some embodiments, the polymer solution in the mold is not degassed.

### Addition of Solid Particulates

In embodiments of the processes described herein, a step in the process is the addition of a solid particulate to the top of the polymer solution in the mold. In processes described herein, the solid particulate is insoluble in the polymer solution, but soluble in the washing liquid. In some embodiments, the solid particulate is sodium chloride, potassium chloride, or sugar. In some embodiments, the solid particulate is sodium chloride or sugar. In some embodiments, the solid particulate is sodium chloride. In some embodiments, the solid particulate is potassium chloride. In some embodiments, the solid particulate is sugar. In some embodiments, the solid particulate is a salt soluble in the washing liquid and insoluble in DMSO and/or THF. In some embodiments, the solid particulate is a salt soluble in the washing liquid and insoluble in DMSO and THF. In some embodiments, the solid particulate is a salt soluble in the washing liquid and insoluble in DMSO or THF. In some embodiments, the solid particulate is a salt soluble in the washing liquid and insoluble in DMSO and soluble in THF. In some embodiments, the solid particulate is a salt soluble in the washing liquid and soluble in DMSO and insoluble in THF. In some embodiments, the solid particulate is a salt soluble in the washing liquid and minimally soluble in DMSO and/or THF.

In some embodiments, the washing liquid is water. In some embodiments, the solid particulate is a salt soluble in water and insoluble in DMSO and/or THF. In some embodiments, the solid particulate is a salt soluble in water and insoluble in DMSO and THF. In some embodiments, the solid particulate is a salt soluble in water and insoluble in DMSO or THF. In some embodiments, the solid particulate is a salt soluble in water and insoluble in DMSO and soluble in THF. In some embodiments, the solid particulate is a salt soluble in water and soluble in DMSO and insoluble in THF. In some embodiments, the solid particulate is a salt soluble in water and minimally soluble in DMSO and/or THF.

In some embodiments, a solid particulate is added to the top of the polymer solution in the mold in a ratio of 10-250 w/v. In some embodiments, a solid particulate is added to the top of the polymer solution in the mold in a ratio of 10-200 w/v. In some embodiments, a solid particulate is added to the top of the polymer solution in the mold in a ratio of 20-200 w/v. In some embodiments, a solid particulate is added to the top of the polymer solution in the mold in a ratio of 20-150 w/v. In some embodiments, a solid particulate is added to the top of the polymer solution in the mold in a ratio of 20-100 w/v. In some embodiments, a solid particulate is added to the top of the polymer solution in the mold in a ratio of 10-250 w/v, wherein the solid particulate is salt. In some embodiments, a solid particulate is added to the top of the polymer solution in the mold in a ratio of 10-200 w/v, wherein the solid particulate is salt. In some embodiments, a solid particulate is added to the top of the polymer solution in the mold in a ratio of 20-200 w/v, wherein the solid particulate is salt. In some embodiments, a solid particulate is added to the top of the polymer solution in the mold in a ratio of 20-150 w/v, wherein the solid particulate is salt. In some embodiments, a solid particulate is added to the top of the polymer solution in the mold in a ratio of 20-100 w/v, wherein the solid particulate is salt. In some embodiments, a solid particulate is added to the top of the polymer solution in the mold in a ratio of 10-250 w/v, wherein the solid particulate is sugar. In some embodiments, a solid particulate is added to the top of the polymer solution in the mold in a ratio of 10-200 w/v, wherein the solid particulate is sugar. In some embodiments, a solid particulate is added to the top of the polymer solution in the mold in a ratio of 20-200 w/v, wherein the solid particulate is sugar. In some embodiments, a solid particulate is added to the top of the polymer solution in the mold in a ratio of 20-150 w/v, wherein the solid particulate is sugar. In some embodiments, a solid particulate is added to the top of the polymer solution in the mold in a ratio of 20-100 w/v, wherein the solid particulate is sugar.

In some embodiments, the solid particulate particle size is between about 5-500 μm. In some embodiments, the solid particulate particle size is between about 5-450 μm. In some embodiments, the solid particulate particle size is between about 5-400 μm. In some embodiments, the solid particulate particle size is between about 5-350 μm. In some embodiments, the solid particulate particle size is between about 5-300 μm. In some embodiments, the solid particulate particle size is between about 5-250 μm. In some embodiments, the solid particulate particle size is between about 5-500 μm, wherein the solid particulate is salt. In some embodiments, the solid particulate particle size is between about 5-450 μm, wherein the solid particulate is salt. In some embodiments, the solid particulate particle size is between about 5-400 μm, wherein the solid particulate is salt. In some embodiments, the solid particulate particle size is between about 5-350 μm, wherein the solid particulate is salt. In some embodiments, the solid particulate particle size is between about 5-300 μm, wherein the solid particulate is salt. In some embodiments, the solid particulate particle size is between about 5-250 μm, wherein the solid particulate is salt. In some embodiments, the solid particulate particle size is between about 5-500 μm, wherein the solid particulate is sugar. In some embodiments, the solid particulate particle size is between about 5-450 μm, wherein the solid particulate is sugar. In some embodiments, the solid particulate particle size is between about 5-400 μm, wherein the solid particulate is sugar. In some embodiments, the solid particulate particle size is between about 5-350 μm, wherein the solid particulate is sugar. In some embodiments, the solid particulate particle size is between about 5-300 μm, wherein the solid particulate is sugar. In some embodiments, the solid particulate particle size is between about 5-250 μm, wherein the solid particulate is sugar.

Washing Polymer Mixture in Mold with a Washing Liquid

In embodiments of the processes described herein, a step in the process is the washing out of the solid particulates in the polymer mixture in the mold with a washing liquid. In some embodiments of the processes described herein, following the addition of the solid particulate to the top of the polymer solution in the mold and before washing the resulting polymer mixture in the mold with the washing liquid, the solvent is removed under vacuum. In some embodiments, following the addition of the solid particulate to the top of the polymer solution in the mold and before washing the resulting polymer mixture in the mold with the washing liquid, the solvent is removed in a vacuum oven at about 30-90° C. In some embodiments, following the addition of the solid particulate to the top of the polymer solution in the mold and before washing the resulting polymer mixture in the mold with the washing liquid, the solvent is removed in a vacuum oven at about 30-70° C. In some embodiments, following the addition of the solid particulate to the top of the polymer solution in the mold and before washing the resulting polymer mixture in the mold with the washing liquid, the solvent is removed in a vacuum oven at about 30-50° C. In some embodiments, following the addition of the solid particulate to the top of the polymer solution in the mold and before washing the resulting polymer mixture in the mold with the washing liquid, the solvent is removed in a vacuum oven at about 30-90° C. for at least 12 hours. In some embodiments, following the addition of the solid particulate to the top of the polymer solution in the mold and before washing the resulting polymer mixture in the mold with the washing liquid, the solvent is removed in a vacuum oven at about 30-70° C. for at least 12 hours. In some embodiments, following the addition of the solid particulate to the top of the polymer solution in the mold and before washing the resulting polymer mixture in the mold with the washing liquid, the solvent is removed in a vacuum oven at about 30-50° C. for at least 12 hours. In some embodiments, following the addition of the solid particulate to the top of the polymer solution in the mold and before washing the resulting polymer mixture in the mold with the washing liquid, the solvent is removed in a vacuum oven at about 30-90° C. for at least 6 hours. In some embodiments, following the addition of the solid particulate to the top of the polymer solution in the mold and before washing the resulting polymer mixture in the mold with the washing liquid, the solvent is removed in a vacuum oven at about 30-70° C. for at least 6 hours. In some embodiments, following the addition of the solid particulate to the top of the polymer solution in the mold and before washing the resulting polymer mixture in the mold with the washing liquid, the solvent is removed in a vacuum oven at about 30-50° C. for at least 6 hours.

In some embodiments, washing the polymer mixture in the mold with a washing liquid comprises immersing the mold in a washing liquid. In some embodiments, washing the polymer mixture in the mold with a washing liquid comprises immersing the mold in the washing liquid, wherein the washing liquid temperature is at room temperature. In some embodiments, washing the polymer mixture in the mold with a washing liquid comprises immersing the mold in the washing liquid, wherein the washing liquid temperature is from about 30-60° C.

In some embodiments of the processes described herein, the washing liquid is water. In embodiments of the processes described herein, a step in the process is the washing out of the solid particulates in the polymer mixture in the mold with water. In some embodiments of the processes described herein, following the addition of the solid particulate to the top of the polymer solution in the mold and before washing the resulting polymer mixture in the mold with water, the solvent is removed under vacuum. In some embodiments, following the addition of the solid particulate to the top of the polymer solution in the mold and before washing the resulting polymer mixture in the mold with water, the solvent is removed in a vacuum oven at about 30-90° C. In some embodiments, following the addition of the solid particulate to the top of the polymer solution in the mold and before washing the resulting polymer mixture in the mold with water, the solvent is removed in a vacuum oven at about 30-70° C. In some embodiments, following the addition of the solid particulate to the top of the polymer solution in the mold and before washing the resulting polymer mixture in the mold with water, the solvent is removed in a vacuum oven at about 30-50° C. In some embodiments, following the addition of the solid particulate to the top of the polymer solution in the mold and before washing the resulting polymer mixture in the mold with water, the solvent is removed in a vacuum oven at about 30-90° C. for at least 12 hours. In some embodiments, following the addition of the solid particulate to the top of the polymer solution in the mold and before washing the resulting polymer mixture in the mold with water, the solvent is removed in a vacuum oven at about 30-70° C. for at least 12 hours. In some embodiments, following the addition of the solid particulate to the top of the polymer solution in the mold and before washing the resulting polymer mixture in the mold with water, the solvent is removed in a vacuum oven at about 30-50° C. for at least 12 hours. In some embodiments, following the addition of the solid particulate to the top of the polymer solution in the mold and before washing the resulting polymer mixture in the mold with water, the solvent is removed in a vacuum oven at about 30-90° C. for at least 6 hours. In some embodiments, following the addition of the solid particulate to the top of the polymer solution in the mold and before washing the resulting polymer mixture in the mold with water, the solvent is removed in a vacuum oven at about 30-70° C. for at least 6 hours. In some embodiments, following the addition of the solid particulate to the top of the polymer solution in the mold and before washing the resulting polymer mixture in the mold with water, the solvent is removed in a vacuum oven at about 30-50° C. for at least 6 hours.

In some embodiments, washing the polymer mixture in the mold with water comprises immersing the mold in water. In some embodiments, washing the polymer mixture in the mold with water comprises immersing the mold in water, wherein the water temperature is at room temperature. In some embodiments, washing the polymer mixture in the mold with water comprises immersing the mold in water, wherein the water temperature is from about 30-60° C.

### Addition of PET Mesh or Membrane to Top of Mold

In some embodiments of the processes described herein, an optional step in the process is the attachment of a mesh or membrane to the foam as part of the casting process. In some embodiments, a PET mesh is attached to the foam as part of the casting process. In some embodiments, after adding the solid particulate to the top of the polymer solution in the mold and before washing the resulting polymer mixture in the mold with a washing liquid, a PET mesh is placed on top of the mold and the mold is heated at 30-90° C. for up to 1 hour. In some embodiments, after adding the solid particulate to the top of the polymer solution in the mold and before washing the resulting polymer mixture in the mold with a washing liquid, a preheated PET mesh is placed on top of the mold and the mold is heated at 30-90° C. for up to 1 hour. In some embodiments, the washing liquid is water. In some embodiments, after adding the solid particulate to the top of the polymer solution in the mold and before washing the resulting polymer mixture in the mold with water, a PET mesh is placed on top of the mold and the mold is heated at 30-90° C. for up to 1 hour. In some embodiments, after adding the solid particulate to the top of the polymer solution in the mold and before washing the resulting polymer mixture in the mold with water, a preheated PET mesh is placed on top of the mold and the mold is heated at 30-90° C. for up to 1 hour. In some embodiments wherein a PET mesh is included in the casting process, the foam is used as a transcatheter heart valve skirt.

### Properties of the Mold and Foam

In some embodiments, the mold is a silicon mold, a Teflon mold, an aluminum mold, a stainless steel mold, or a glass mold. In some embodiments, the mold is a silicon mold, a Teflon mold, or an aluminum mold. In some embodiments, the mold is a Teflon mold. In some embodiments, the mold is an aluminum mold. In some embodiments, the mold is a stainless steel mold. In some embodiments, the mold is a glass mold. In some embodiments, a mother mold of the desired pattern for casting if designed by 3D modelling software, for example Solidworks, and printed using a 3D printer. In some embodiments, a negative of the desired pattern is made using a silicon mold from an appropriate castable silicone, for example PinkySil®.

In embodiments, the foam is cast into a predetermined shape at a based on the shape of the mold. In some embodiments, the foam is cast into a predetermined shape at a millimeter scale based on the shape of the mold. In some embodiments, the shape of the mold is selected from a triangular pyramid, square pyramid, oval, partial oval, spherical, partially spherical, rhomboid, diamond, partial diamond, and a customized 3-dimensional shape; or a combination thereof. In some embodiments, the shape of the mold is a triangular pyramid. In some embodiments, the shape of the mold is a square pyramid. In some embodiments, the shape of the mold is an oval. In some embodiments, the shape of the mold is a partial oval. In some embodiments, the shape of the mold is spherical. In some embodiments, the shape of the mold is partially spherical. In some embodiments, the shape of the mold is a rhomboid. In some embodiments, the shape of the mold is diamond. In some embodiments, the shape of the mold is partial diamond. In some embodiments, the shape of the mold is a customized 3-dimensional shape. In some embodiments, the shape of the mold is a combination of one or more shapes selected from a triangular pyramid, square pyramid, oval, partial oval, spherical, partially spherical, rhomboid, diamond, partial diamond, and a customized 3-dimensional shape.

In some embodiments, the foams prepared by the processes described herein have a controlled porosity. In some embodiments, the foam has a uniform porosity. In some embodiments, the foam has a variable porosity. In some embodiments, the foam has an open pore structure, a closed pore structure, or a combination. In some embodiments, the foam has a porosity of about 5-500 μm. In some embodiments, the foam has a porosity of about 5-500 μm. In some embodiments, the foam has a porosity of about 5-450 μm. In some embodiments, the foam has a porosity of about 5-400 μm. In some embodiments, the foam has a porosity of about 5-350 μm. In some embodiments, the foam has a porosity of about 5-300 μm. In some embodiments, the foam has a porosity of about 5-250 μm. In some embodiments, the foam has a porosity of about 5-200 μm.

### Uses

In some embodiments, the foams prepared by the processes described herein are used as a foam-based sealing device, sealing cuff, or sealing skirt for endovascular prostheses. In some embodiments, the foams prepared by the processes described herein are used as a sealing device. In some embodiments, the foams prepared by the processes described herein are used as a sealing cuff. In some embodiments, the foams prepared by the processes described herein are used as a sealing skirt for endovascular prostheses. In some embodiments, the foams prepared by the processes described herein are used as foam-based expandable sealing skirts for use with an endovascular prosthesis in a human patient. In some embodiments, the endovascular prosthesis is a transcatheter valve implantation device. In some embodiments, the endovascular prosthesis is a transcatheter aortic valve implantation device. In some embodiments, the endovascular prosthesis is an endovascular stent graft.

Unless otherwise defined, all technical terms used herein have the same meaning as commonly understood by one of ordinary skill in the art to which this disclosure belongs. As used in this specification and the appended claims, the singular forms “a,” “an,” and “the” include plural references unless the context clearly dictates otherwise. Any reference to “or” herein is intended to encompass “and/or” unless otherwise stated. As used in this specification and the claims, unless otherwise stated, the term “about,” and “approximately” refers to variations of less than or equal to +/−1%, +/−2%, +/−3%, +/−4%, +/−5%, +/−6%, +/−7%, +/−8%, +/−9%, +/−10%, +/−11%, +/−12%, +/−14%, +/−15%, or +/−20% of the numerical value depending on the embodiment. As a non-limiting example, about 100 meters represents a range of 95 meters to 105 meters (which is +/−5% of 100 meters), 90 meters to 110 meters (which is +/−10% of 100 meters), or 85 meters to 115 meters (which is +/−15% of 100 meters) depending on the embodiments.

### EXAMPLES

### Example 1: Foam Casting Procedure Using DMSO

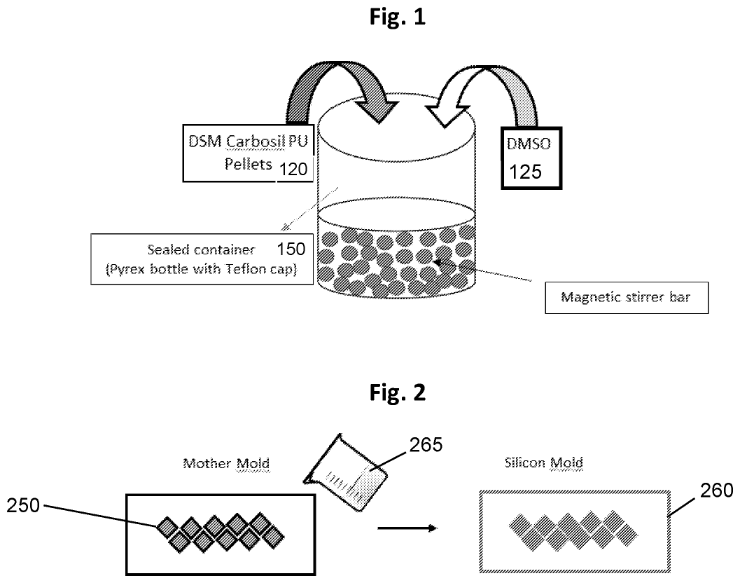

As depicted by FIG. 1, DSM Carbosil 80A and 55D Pellets 120 are stored in a desiccator at approximately room temperature, at moisture content ranging from of 30-33%. Silica gel beads are used to absorb the moisture in a desiccant cabinet.

Polymer solutions are made by dissolving polyurethane (PU) pellets 120 in DMSO 125 with around 5-25% TDS. The pellets were dissolved by adding solvent on top of pellets in a Pyrex bottle 150 with Teflon cap stirring on a magnetic stirrer at room temperature. Depending on the polymer concentration, complete dissolution takes 20-80 hours.

As depicted by FIG. 2, a mother mold 250 of the desired pattern that needs to be cast is first designed by a 3D modelling software, e.g. Solidworks, and printed using a 3D printer. Then, a negative of the desired pattern is made using a silicon mold 260 from an appropriate castable silicone 265 e.g. PinkySil®.

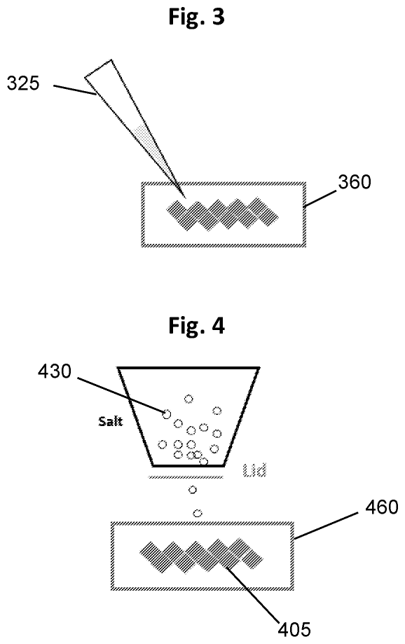

The silicon mold is preheated at temperature between 40-100° C. for at least half an hour. The polymer solution which has been stored at room temperature is added to the silicon mold to fill the mold until it overflows. The amount of solution depends on the mold size and is optimized for each mold. The solution may be pre-heated, if necessary. In some embodiments, as depicted in FIG. 3, a pipette 325 is used to add the polymer solution to the silicon mold 360.

The solution in the silicon mold may be degassed in a vacuum oven at 30-90° C. for up to 1 hour (vacuum up to 300 mbar). As depicted by FIG. 4, salt 430 is weighed and then added on top of the optionally degassed solution 405 in the mold 460. The ratio of the salt to solution is optimized for different molds. This step can be done either manually or using an automated means.

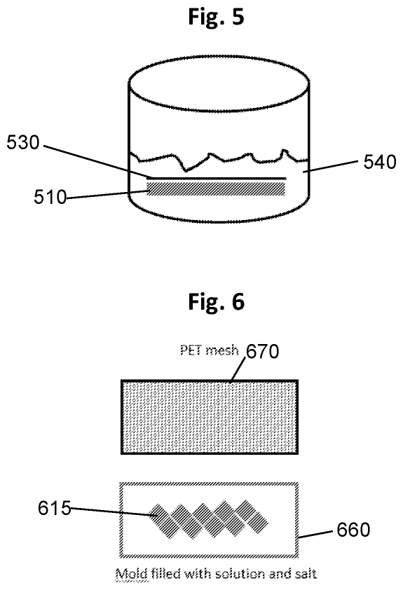

As depicted in FIG. 5, the sample is removed from oven/vacuum oven and sunk in hot tap water 540 in order to wash out the DMSO. Washing also leaches out the salt 530 so that the remaining polymer material forms the scaffold 510 of the foam. Upon removal of the salt crystals all that remains is the hardened polymer with open holes/pores where the salt once was.

Foams, now in final form, are carefully removed from the silicon molds and are stored in tap water in 120 mL containers.

### Example 2: Foam Casting Procedure Using DMSO Including Use of PET Mesh

In some embodiments, as depicted in FIG. 6, wherein the foam is used as a transcatheter heart valve skirt, the use of a PET mesh 670 is included in the casting process. Following the addition of salt to the polymer solution 615 in the mold 660 as outlined in Example 1, PET mesh is cut as the same size of the mold and then it is preheated in oven at 50-100° C. for at least half an hour. The PET mesh is placed on top of the mold (FIG. 6) and the mold/PET mesh sample is heated at 30-90° C. for up to an hour. The sample is removed from oven/vacuum oven and sunk in hot tap water (FIG. 5) in order to wash out the DMSO. Washing also leaches out the salt so that the remaining polymer material forms the scaffold of the foam. Upon removal of the salt crystals, what remains is the hardened polymer foam with the PET mesh attached to the foam surface.

### Example 3: Foam Casting Procedure Using THF

DSM Carbosil 80A and 55D Pellets are stored in a desiccator at approximately room temperature, at moisture content ranging from of 30-33%. Silica gel beads are used to absorb the moisture in a desiccant cabinet.

Two polymer solutions are made by dissolving polyurethane (PU) pellets in THF; a higher concentration solution in the range of 8-15% (w/v) and a lower concentration solution in the range of 0.5-6% (w/v). The pellets are dissolved by adding solvent on top of pellets in a Pyrex bottle with Teflon cap stirring on a magnetic stirrer at room temperature.

A mother mold of the desired pattern that needs to be cast is first designed by a 3D modelling software, e.g. Solidworks, and printed using a 3D printer. Then, a negative of the desired pattern is made using a silicon mold from an appropriate castable silicone, e.g. PinkySil®.

A thin layer of PU is created on the silicon mold using the lower concentration PU THF solution. When the thin layer of PU is dried, salt is added to the silicon mold to fill the spaces completely.

The higher concentration polymer THF solution is added to the silicon mold to cover the salt completely (the volume of polymer solution is optimised for each design of silicon mold and is kept consistent between samples).

The mold is left under a fumehood for at least 30 minutes so that the polymer solution diffuses into the structure of foam. The sample is covered with a glass beaker to minimize the evaporation of solvent during this time.

The mold is placed in a vacuum oven at 50° C. for a minimum of 12 hours to remove the solvent and obtain a solid structure of foam.

The sample is removed from oven/vacuum oven and washed with water until the salt is fully dissolved and removed.

Foams, now in final form, are carefully removed from the silicon molds and are kept wet in PBS.

### Example 4: Foam Casting Procedure Using THF Including Use of PET Mesh

In some embodiments wherein the foam is used as a transcatheter heart valve skirt, the use of a PET mesh is included in the casting process. Following the addition of salt to the polymer solution in the mold as outlined in Example 3, PET mesh is cut as the same size of the mold and then it is preheated in oven at 50-100° C. for at least half an hour. The PET mesh is placed on top of the mold and an additional layer of the higher concentration polymer THF solution is added to the mold/PET mesh sample to obtain a uniform distribution of polymer solution on the surface. The mold/PET mesh sample is heated at 30-90° C. for up to an hour. The sample is removed from oven/vacuum oven and washed with water until the salt is fully dissolved and removed. Upon removal of the salt crystals, what remains is the hardened polymer foam with the PET mesh attached to the foam surface.

### Example 5: Foam Casting Procedure Using Co-Solvents

Foams are manufactured using the processes outlined in Examples 1-4, but a combination on DMSO and THF is used to prepare the polymer solution. The ratio of DMSO to THF can be varied. Use of various co-solvent ratios allows for solutions with higher concentration of polymer and lower viscosity and as a result foams with different mechanical properties.

While preferred embodiments disclosed herein have been shown and described herein, it will be obvious to those skilled in the art that such embodiments are provided by way of example only. Numerous variations, changes, and substitutions will now occur to those skilled in the art without departing from the disclosure herein. It should be understood that various alternatives to the embodiments of the casting processes described herein may be employed in practicing the casting processes herein.

### Currently Preferred Embodiments

1. A process for the preparation of a foam, the process comprising:
a) dissolving a polymer material in a solvent to form a polymer solution;
b) adding the polymer solution to a mold, and optionally degassing the polymer solution in the mold;
c) adding a solid particulate to the top of the polymer solution in the mold; and
d) washing the resulting polymer mixture in the mold with a washing liquid;

wherein the solid particulate is insoluble in the polymer solution and soluble in the washing liquid, and wherein after washing the polymer mixture with the washing liquid, the remaining polymer material in the mold forms the foam and the shape of the foam is defined by the shape of the mold.

2. The process of paragraph 1, further comprising forming a thin layer of polymer material on the mold before adding the polymer solution to the mold.

3. The process of paragraph 1 or 2, wherein the concentration of the polymer solution is from 0.5%-25.0% (w/v).

4. The process of any one of paragraphs 1-3, wherein after adding the solid particulate to the top of the polymer solution in the mold and before washing the resulting polymer mixture in the mold with the washing liquid, further comprising placing a preheated PET mesh on top of the mold and heating the mold at 30-90° C. for up to 1 hour.

5. The process of any one of paragraphs 1-4, wherein the mold is preheated to a temperature from about 40-100° C. prior to the addition of the polymer solution to the mold.

6. The process of any one of paragraphs 1-5, wherein the mold is preheated to a temperature from about 40-100° C. for at least 20 minutes.

7. The process of any one of paragraphs 1-6, wherein the polymer solution is at room temperature, or preheated to a temperature from about 30-70° C., upon adding the polymer solution to the mold.

8. The process of any one of paragraphs 1-7, wherein the polymer solution is added to the mold until it overflows.

9. The process of any one of paragraphs 1-8, wherein the polymer solution in the mold is degassed.

10. The process of any one of paragraphs 1-9, wherein the polymer solution in the mold is degassed in a vacuum oven.

11. The process of any one of paragraphs 1-10, wherein the polymer solution in the mold is degassed in a vacuum oven at about 100 mbar-500 mbar.

12. The process of any one of paragraphs 1-11, wherein the polymer solution in the mold is degassed in a vacuum oven at about 30-90° C.

13. The process of any one of paragraphs 1-12, wherein the polymer solution in the mold is degassed in a vacuum oven at about 30-90° C. for up to 1 hour.

14. The process of any one of paragraphs 1-9, wherein the polymer solution in the mold is not degassed.

15. The process of any one of paragraphs 1-14, wherein the solid particulate is added to the top of the polymer solution in the mold in a ratio of 10-250 w/v.

16. The process of any one of paragraphs 1-15, wherein the solid particulate particle size is between about 5-500 μm.

17. The process of any one of paragraphs 1-16, wherein following the addition of the solid particulate to the top of the polymer solution in the mold and before washing the resulting polymer mixture in the mold with the washing liquid, the solvent is removed under vacuum.

18. The process of any one of paragraphs 1-17, wherein following the addition of the solid particulate to the top of the polymer solution in the mold and before washing the resulting polymer mixture in the mold with the washing liquid, the solvent is removed in a vacuum oven at about 30-90° C.

19. The process of any one of paragraphs 1-18, wherein following the addition of the solid particulate to the top of the polymer solution in the mold and before washing the resulting polymer mixture in the mold with the washing liquid, the solvent is removed in a vacuum oven at about 30-90° C. for at least 12 hours.

20. The process of any one of paragraphs 1-19, wherein washing the resulting polymer mixture in the mold with the washing liquid comprises immersing the mold in the washing liquid.

21. The process of any one of paragraphs 1-20, wherein washing the resulting polymer mixture in the mold with the washing liquid comprises immersing the mold in the washing liquid, wherein the washing liquid temperature is from about 30-60° C.

22. The process of any one of paragraphs 1-21, wherein the washing liquid is water.

23. The process of any one of paragraphs 1-22, wherein the solvent is selected from dimethyl sulfoxide (DMSO), tetrahydrofuran (THF), dimethylformamide (DMF), dimethylacetamide (DMAc), and a combination thereof.

24. The process of any one of paragraphs 1-23, wherein the solvent is DMSO.

25. The process of any one of paragraphs 1-23, wherein the solvent is THF.

26. The process of any one of paragraphs 1-23, wherein the solvent is DMF.

27. The process of any one of paragraphs 1-23, wherein the solvent is DMAc.

28. The process of any one of paragraphs 1-23, wherein the solvent is a combination of DMSO and THF.

29. The process of any one of paragraphs 1-28, wherein the polymer material is pre-polymerized polymer pellets.

30. The process of any one of paragraphs 1-29, wherein the polymer material is polyurethane pellets.

31. The process of any one of paragraphs 1-30, wherein the solid particulate is sodium chloride.

32. The process of any one of paragraphs 1-30, wherein the solid particulate is sugar. 33. The process of any one of paragraphs 1-32, wherein the foam has a controlled porosity.

34. The process of any one of paragraphs 1-32, wherein the foam has a uniform porosity.

35. The process of any one of paragraphs 1-32, wherein the foam has a variable porosity.

36. The process of any one of paragraphs 1-35, wherein the foam has an open pore structure, a closed pore structure, or a combination.

37. The process of any one of paragraphs 1-36, wherein the foam has a porosity of about 5-500 μm.

38. The process of any one of paragraphs 1-37, wherein the foam is cast into a predetermined shape at a millimeter scale based on the shape of the mold.

39. The process of paragraphs 38, wherein the shape of the mold is selected from a triangular pyramid, square pyramid, oval, partial oval, spherical, partially spherical, rhomboid, diamond, partial diamond, and a customized 3-dimensional shape; or a combination thereof.

40. The process of any one of paragraphs 1-39, wherein the mold is a silicon mold, a Teflon mold, or an aluminum mold.

41. The process of any one of paragraphs 1-40, wherein the foam is used as a foam-based expandable sealing skirt for use with an endovascular prosthesis in a human patient.

42. The process of paragraph 41, wherein the endovascular prosthesis is a transcatheter valve implantation device.

43. The process of paragraph 41, wherein the endovascular prosthesis is a transcatheter aortic valve implantation device.

44. The process of paragraph 41, wherein the endovascular prosthesis is an endovascular stent graft.

45. The process of any one of paragraphs 1-44, wherein the concentration of the polymer solution is from 0.5%-20.0% (w/v).

46. The process of any one of paragraphs 1-44, wherein the concentration of the polymer solution is from 0.5%-15.0% (w/v).

47. The process of any one of paragraphs 1-44, wherein the concentration of the polymer solution is from 0.5%-10.0% (w/v).

48. The process of any one of paragraphs 1-44, wherein the concentration of the polymer solution is from 5.0%-25.0% (w/v).

49. The process of any one of paragraphs 1-44, wherein the concentration of the polymer solution is from 1.0%-25.0% (w/v).

50. The process of any one of paragraphs 1-44, wherein the concentration of the polymer solution is from 2.0%-25.0% (w/v).

51. The process of any one of paragraphs 1-44, wherein the concentration of the polymer solution is from 3.0%-25.0% (w/v).

52. The process of any one of paragraphs 1-44, wherein the concentration of the polymer solution is from 4.0%-25.0% (w/v).

53. The process of any one of paragraphs 1-44, wherein the concentration of the polymer solution is from 5.0%-20.0% (w/v).

54. The process of any one of paragraphs 1-44, wherein the concentration of the polymer solution is from 5.0%-15.0% (w/v).

55. The process of any one of paragraphs 1-54, wherein the polymer solution in the mold is degassed in a vacuum oven at about 100 mbar-400 mbar.

56. The process of any one of paragraphs 1-54, wherein the polymer solution in the mold is degassed in a vacuum oven at about 100 mbar-300 mbar.

57. The process of any one of paragraphs 1-56, wherein the solid particulate is added to the top of the polymer solution in the mold in a ratio of 10-200 w/v.

58. The process of any one of paragraphs 1-56, wherein the solid particulate is added to the top of the polymer solution in the mold in a ratio of 20-200 w/v.

59. The process of any one of paragraphs 1-56, wherein the solid particulate is added to the top of the polymer solution in the mold in a ratio of 20-150 w/v.

60. The process of any one of paragraphs 1-56, wherein the solid particulate is added to the top of the polymer solution in the mold in a ratio of 20-100 w/v.

61. The process of any one of paragraphs 1-60, wherein the solid particulate particle size is between about 5-450 μm.

62. The process of any one of paragraphs 1-60, wherein the solid particulate particle size is between about 5-400 μm.

63. The process of any one of paragraphs 1-60, wherein the solid particulate particle size is between about 5-350 μm.

64. The process of any one of paragraphs 1-60, wherein the solid particulate particle size is between about 5-300 μm.

65. The process of any one of paragraphs 1-60, wherein the solid particulate particle size is between about 5-250 μm.

66. The process of any one of paragraphs 1-65, wherein the foam has a porosity of about 5-500 μm.

67. The process of any one of paragraphs 1-36, wherein the foam has a porosity of about 5-450 μm.

68. The process of any one of paragraphs 1-36, wherein the foam has a porosity of about 5-400 μm.

69. The process of any one of paragraphs 1-36, wherein the foam has a porosity of about 5-350 μm.

70. The process of any one of paragraphs 1-36, wherein the foam has a porosity of about 5-300 μm.

71. The process of any one of paragraphs 1-36, wherein the foam has a porosity of about 5-250 μm.

What is claimed is:

1. A process for the preparation of a foam, the process comprising:
a) dissolving a polymer material in a solvent to form a polymer solution;
b) adding the polymer solution to a mold, and optionally degassing the polymer solution in the mold;
c) adding a solid particulate to the top of the polymer solution in the mold; and
d) washing the resulting polymer mixture in the mold with a washing liquid;
wherein the solid particulate is insoluble in the polymer solution and soluble in the washing liquid, and wherein after washing the polymer mixture with the washing liquid, the remaining polymer material in the mold forms the foam and the shape of the foam is defined by the shape of the mold.

2. The process of claim 1, further comprising forming a thin layer of polymer material on the mold before adding the polymer solution to the mold.

3. The process of claim 1, wherein the concentration of the polymer solution is from 0.5%-25.0% (w/v).

4. The process of claim 1, wherein after adding the solid particulate to the top of the polymer solution in the mold and before washing the resulting polymer mixture in the mold with the washing liquid, further comprising placing a preheated PET mesh on top of the mold and heating the mold at 30-90° C. for up to 1 hour.

5. The process of claim 1, wherein the mold is preheated to a temperature from about 40-100° C. prior to the addition of the polymer solution to the mold.

6. The process of claim 1, wherein the mold is preheated to a temperature from about 40-100° C. for at least 20 minutes.

7. The process of claim 1, wherein the polymer solution is at room temperature, or preheated to a temperature from about 30-70° C., upon adding the polymer solution to the mold.

8. The process of claim 1, wherein the polymer solution is added to the mold until it overflows.

9. The process of claim 1, wherein the polymer solution in the mold is degassed.

10. The process of claim 9, wherein the polymer solution in the mold is degassed in a vacuum oven.

11. The process of claim 10, wherein the polymer solution in the mold is degassed in a vacuum oven at about 100 mbar-500 mbar.

12. The process of claim 10, wherein the polymer solution in the mold is degassed in a vacuum oven at about 30-90° C.

13. The process of claim 10, wherein the polymer solution in the mold is degassed in a vacuum oven at about 30-90° C. for up to 1 hour.

14. The process of claim 1, wherein the polymer solution in the mold is not degassed.

15. The process of claim 1, wherein the solid particulate is added to the top of the polymer solution in the mold in a ratio of 10-250 w/v.

16. The process of claim 15, wherein the solid particulate particle size is between about 5-500 μm.

17. The process of claim 15, wherein following the addition of the solid particulate to the top of the polymer solution in the mold and before washing the resulting polymer mixture in the mold with the washing liquid, the solvent is removed under vacuum.

18. The process of claim 15, wherein following the addition of the solid particulate to the top of the polymer solution in the mold and before washing the resulting polymer mixture in the mold with the washing liquid, the solvent is removed in a vacuum oven at about 30-90° C.

19. The process of claim 15, wherein following the addition of the solid particulate to the top of the polymer solution in the mold and before washing the resulting polymer mixture in the mold with the washing liquid, the solvent is removed in a vacuum oven at about 30-90° C. for at least 12 hours.

20. The process of claim 1, wherein washing the resulting polymer mixture in the mold with the washing liquid comprises immersing the mold in the washing liquid.

21. The process of claim 1, wherein washing the resulting polymer mixture in the mold with the washing liquid comprises immersing the mold in the washing liquid, wherein the washing liquid temperature is from about 30-60° C.

22. The process claim 21, wherein the washing liquid is water.

23. The process of claim 1, wherein the solvent is selected from dimethyl sulfoxide (DMSO), tetrahydrofuran (THF), dimethylformamide (DMF), dimethylacetamide (DMAc), and a combination thereof.

24. The process of claim 23, wherein the solvent is DMSO.

25. The process of claim 23, wherein the solvent is THF.

26. The process of claim 23, wherein the solvent is DMF.

27. The process of claim 23, wherein the solvent is DMAc.

28. The process of claim 23, wherein the solvent is a combination of DMSO and THF.

29. The process of claim 1, wherein the polymer material is pre-polymerized polymer pellets.

30. The process of claim 29, wherein the polymer material is polyurethane pellets.

31. The process of claim 1, wherein the solid particulate is sodium chloride.

32. The process of claim 1, wherein the solid particulate is sugar.

33. The process of claim 1, wherein the foam has a controlled porosity.

34. The process of claim 1, wherein the foam has a uniform porosity.

35. The process of claim 1, wherein the foam has a variable porosity.

36. The process of claim 1, wherein the foam has an open pore structure, a closed pore structure, or a combination.

37. The process of claim 1, wherein the foam has a porosity of about 5-500 μm.

38. The process of claim 1, wherein the foam is cast into a predetermined shape at a millimeter scale based on the shape of the mold.

39. The process of claim 38, wherein the shape of the mold is selected from a triangular pyramid, square pyramid, oval, partial oval, spherical, partially spherical, rhomboid, diamond, partial diamond, and a customized 3-dimensional shape; or a combination thereof.

40. The process of claim 1, wherein the mold is a silicon mold, a Teflon mold, or an aluminum mold.

41. The process of claim 1, wherein the foam is used as a foam-based expandable sealing skirt for use with an endovascular prosthesis in a human patient.

42. The process of claim 41, wherein the endovascular prosthesis is a transcatheter valve implantation device.

43. The process of claim 41, wherein the endovascular prosthesis is a transcatheter aortic valve implantation device.

44. The process of claim 41, wherein the endovascular prosthesis is an endovascular stent graft.

45. The process of claim 1, wherein the concentration of the polymer solution is from 0.5%-20.0% (w/v).

46. The process of claim 1, wherein the concentration of the polymer solution is from 0.5%-15.0% (w/v).

47. The process of claim 1, wherein the concentration of the polymer solution is from 0.5%-10.0% (w/v).

48. The process of claim 1, wherein the concentration of the polymer solution is from 5.0%-25.0% (w/v).

49. The process of claim 1, wherein the concentration of the polymer solution is from 1.0%-25.0% (w/v).

50. The process of claim 1, wherein the concentration of the polymer solution is from 2.0%-25.0% (w/v).

51. The process of claim 1, wherein the concentration of the polymer solution is from 3.0%-25.0% (w/v).

52. The process of claim 1, wherein the concentration of the polymer solution is from 4.0%-25.0% (w/v).

53. The process of claim 1, wherein the concentration of the polymer solution is from 5.0%-20.0% (w/v).

54. The process of claim 1, wherein the concentration of the polymer solution is from 5.0%-15.0% (w/v).

55. The process of claim 1, wherein the polymer solution in the mold is degassed in a vacuum oven at about 100 mbar-400 mbar.

56. The process of claim 1, wherein the polymer solution in the mold is degassed in a vacuum oven at about 100 mbar-300 mbar.

57. The process of claim 1, wherein the solid particulate is added to the top of the polymer solution in the mold in a ratio of 10-200 w/v.

58. The process of claim 1, wherein the solid particulate is added to the top of the polymer solution in the mold in a ratio of 20-200 w/v.

59. The process of claim 1, wherein the solid particulate is added to the top of the polymer solution in the mold in a ratio of 20-150 w/v.

60. The process of claim 1, wherein the solid particulate is added to the top of the polymer solution in the mold in a ratio of 20-100 w/v.

61. The process of claim 1, wherein the solid particulate particle size is between about 5-450 μm.

62. The process of claim 1, wherein the solid particulate particle size is between about 5-400 μm.

63. The process of claim 1, wherein the solid particulate particle size is between about 5-350 μm.

64. The process of claim 1, wherein the solid particulate particle size is between about 5-300 μm.

65. The process of claim 1, wherein the solid particulate particle size is between about 5-250 μm.

66. The process of claim 1, wherein the foam has a porosity of about 5-500 μm.

67. The process of claim 1, wherein the foam has a porosity of about 5-450 μm.

68. The process of claim 1, wherein the foam has a porosity of about 5-400 μm.

69. The process of claim 1, wherein the foam has a porosity of about 5-350 μm.

70. The process of claim 1, wherein the foam has a porosity of about 5-300 μm.

71. The process of claim 1, wherein the foam has a porosity of about 5-250 μm.
